(19) United States
(12) Patent Application Publication
(10) Pub. No.: US 2026/0215696 A1
(43) Pub. Date: Jul. 30, 2026

(54) WEARABLE ELECTRONIC DEVICE

(71) Applicant: SAMSUNG ELECTRONICS CO., LTD., Suwon-si (KR)

(72) Inventors: Hongki KIM, Suwon-si (KR); Daegyu KANG, Suwon-si (KR); Younghyun KIM, Suwon-si (KR)

(73) Assignee: SAMSUNG ELECTRONICS CO., LTD., Suwon-si (KR)

(21) Appl. No.: 19/578,429

(22) Filed: Mar. 25, 2026

Related U.S. Application Data
(63) Continuation of application No. PCT/KR2024/014394, filed on Sep. 24, 2024.

(30) Foreign Application Priority Data
Sep. 26, 2023 (KR) 10-2023-0129733
Dec. 12, 2023 (KR) 10-2023-0179909

(51) Int. Cl. A61B 5/024; H04M 1/02

(52) U.S. Cl. CPC A61B 5/02438; H04M 1/0262; H04M 1/0277; H04M 2250/12

(57) ABSTRACT

An electronic device includes: a housing having a ring shape and including an outer housing part and an inner housing part, the inner housing part being coupled to the outer housing part; a circuit board disposed within the housing; at least one optical element disposed within the housing, the at least one optical element being electrically connected to the circuit board; and a plurality of protrusions arranged along an inner circumferential surface of the inner housing part, wherein the at least one optical element faces at least one protrusion of the plurality of protrusions.

### CROSS-REFERENCE TO RELATED APPLICATION

This application is a by-pass continuation application of International Application No. PCT/KR2024/014394, filed on September 24, 2024, which is based on and claims priority to Korean Patent Application No. 10-2023-0129733, filed on September 26, 2023, and Korean Patent Application No. 10-2023-0179909, filed on December 12, 2023, in the Korean Ministry of Intellectual Property, the disclosures of which are incorporated by reference herein in their entireties.

### 1. FIELD

The present disclosure relates to a wearable electronic device.

### 2. DESCRIPTION OF RELATED ART

In the related art, electronic devices have evolved into various forms for user convenience and have been miniaturized to allow users to easily carry the electronic devices. For example, an electronic device may be provided in the form of a ring to be wearable on a user’s finger (or wrist). In addition, as interest in health increases, interest in technologies capable of checking health conditions has also grown.

Accordingly, an electronic device may include sensors for measuring a user’s biometric information and has been developed in various forms to measure and utilize various biometric signals of the human body. Through the measurement of various biometric signals, the electronic device may provide various services for managing or checking the user’s health conditions.

The above-described information may be provided as related art for the purpose of helping to understand the disclosure. No claim or determination is made as to whether any of the foregoing may be applied as prior art with respect to the disclosure.

### SUMMARY

According to an aspect of the disclosure, an electronic device includes: a housing having a ring shape and including an outer housing part and an inner housing part, the inner housing part being coupled to the outer housing part; a circuit board disposed within the housing; at least one optical element disposed within the housing, the at least one optical element being electrically connected to the circuit board; and a plurality of protrusions arranged along an inner circumferential surface of the inner housing part, wherein the at least one optical element faces at least one protrusion of the plurality of protrusions.

A plurality of recess regions may be provided between adjacent protrusions of the plurality of protrusions.

Each protrusion of the plurality of protrusions may include a central portion and an edge portion, and a vertical distance from the central portion to the inner circumferential surface of the inner housing part is greater than a vertical distance from the edge portion to the inner circumferential surface of the inner housing part.

The plurality of protrusions may be integrally formed with the inner housing part .

The plurality of protrusions may be at least partially transparent.

The plurality of protrusions may include: a first protrusion; a second protrusion positioned in a first circumferential direction from the first protrusion and extending from the first protrusion; and a third protrusion positioned in a second circumferential direction and extending from the first protrusion, the second circumferential direction being opposite to the first circumferential direction..

The plurality of protrusions may further include: a fourth protrusion arranged symmetrically with the first protrusion with respect to a center of the housing; a fifth protrusion arranged symmetrically with the second protrusion with respect to the center of the housing and positioned in the first circumferential direction from the fourth protrusion; and a sixth protrusion arranged symmetrically with the third protrusion with respect to the center of the housing and positioned in the second circumferential direction from the fourth protrusion.

Each protrusion among the plurality of protrusions may include a flat surface oriented toward the center of the housing.

Each protrusion among the plurality of protrusions may include a curved surface oriented toward the center of the housing.

The circuit board may include rigid printed circuit boards and flexible printed circuit boards that are alternately arranged.

The electronic device may further include a battery disposed within the housing, and the housing may include a first region and a second region, the battery is disposed in the first region, and the circuit board is disposed in the second region.

The electronic device may further include a processor disposed within the housing, the at least one optical element may include: at least one light emitter configured to emit light through the inner housing part, and at least one light receiver configured to receive light transmitted through or reflected from a user’s finger, and the processor may be configured to obtain at least one biometric data based on a signal detected by the at least one light receiver.

The at least one biometric data may include at least one of oxygen saturation data or heart rate data.

The electronic device may further include at least one blocking member disposed within the housing and configured to absorb or block light.

The at least one light emitter may be configured to emit light in a plurality of wavelength bands, and the plurality of wavelength bands may include a red wavelength band and an infrared wavelength band.

An electronic device according to one or more embodiments of the disclosure may be a wearable smart ring device in the form of a ring, in which a plurality of protrusions formed on one surface of an inner housing forming an inner circumference in contact with the skin and recesses formed between the protrusions constitute an air passage when the device is worn, thereby forming a structure that facilitates attachment and detachment.

### BRIEF DESCRIPTION OF THE DRAWINGS

The above and other aspects, features, and advantages of certain embodiments of the present disclosure are more apparent from the following description taken in conjunction with the accompanying drawings, in which:

FIG. 1 is a block diagram of an electronic device in a network environment, according to an embodiment of the disclosure;

FIG. 2 is a view illustrating examples of use of a wearable electronic device according to an embodiment of the disclosure;

FIG. 3 is a perspective view illustrating a wearable electronic device according to an embodiment of the disclosure;

FIG. 4 is a cross-sectional view of the wearable electronic device according to an embodiment of the disclosure;

FIG. 5 is an exploded perspective view of the wearable electronic device according to an embodiment of the disclosure;

FIG. 6 is a perspective view of a wearable electronic device according to an embodiment of the disclosure;

FIG. 7 is a side view of the wearable electronic device according to an embodiment of the disclosure;

FIGS. 8A and 8B are side views of a wearable electronic device 200 according to an embodiment of the disclosure;

FIGS. 9 and 10 are views illustrating a portion of an internal space of the wearable electronic device 200 according to an embodiment of the disclosure;

FIG. 11 is a side view of a wearable electronic device 200 according to an embodiment of the disclosure; and

FIGS. 12 and 13 are views illustrating a portion of an internal space of the wearable electronic device 200 according to an embodiment of the disclosure.

### DETAILED DESCRIPTION

The embodiments described in the disclosure, and the configurations shown in the drawings, are only examples of embodiments, and various modifications may be made without departing from the scope and spirit of the disclosure.

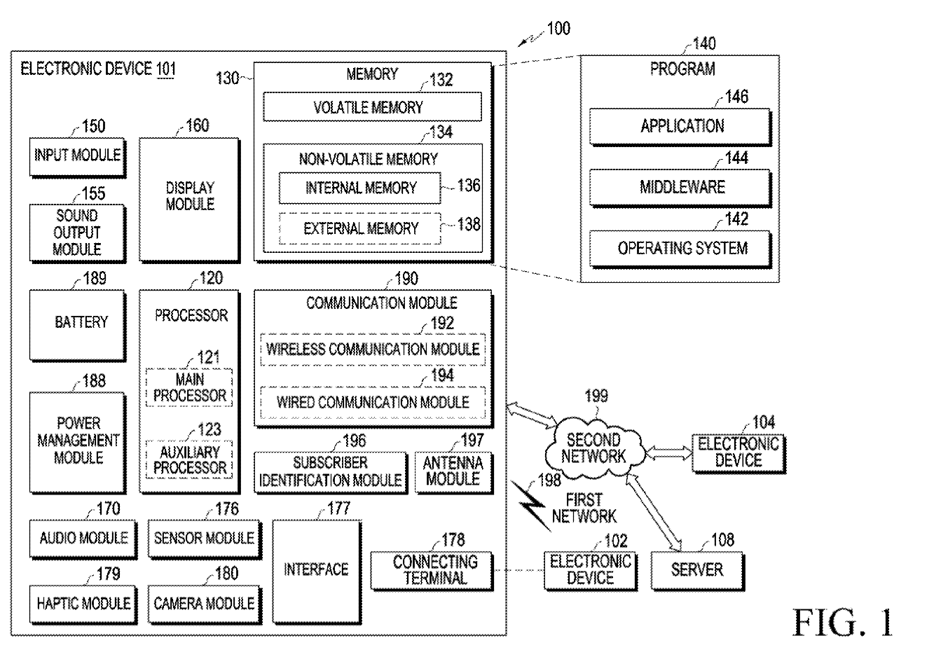

FIG. 1 is a block diagram illustrating an electronic device 101 in a network environment 100 according to various embodiments.

Referring to FIG. 1, the electronic device 101 in the network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In some embodiments, at least one of the components (e.g., the connecting terminal 178) may be omitted from the electronic device 101, or one or more other components may be added in the electronic device 101. In some embodiments, some of the components (e.g., the sensor module 176, the camera module 180, or the antenna module 197) may be implemented as a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to one embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted Boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the intensity of force incurred by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to one embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth^{TM}, wireless-fidelity (Wi-Fi) direct, or infrared data association (IrDA)) or the second network 199 (e.g., a long-range communication network, such as a legacy cellular network, a 5G network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a 4G network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the mmWave band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20Gbps or more) for implementing eMBB, loss coverage (e.g., 164dB or less) for implementing mMTC, or U-plane latency (e.g., 0.5ms or less for each of downlink (DL) and uplink (UL), or a round trip of 1ms or less) for implementing URLLC.

The antenna module 197 may transmit or receive a signal or power to or from the outside (e.g., the external electronic device) of the electronic device 101. According to an embodiment, the antenna module 197 may include an antenna including a radiating element composed of a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to various embodiments, the antenna module 197 may form a mmWave antenna module. According to an embodiment, the mmWave antenna module may include a printed circuit board, a RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In another embodiment, the external electronic device 104 may include an internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

The electronic device according to various embodiments may be one of various types of electronic devices. The electronic devices may include, for example, a portable communication device (e.g., a smartphone), a computer device, a portable multimedia device, a portable medical device, a camera, a wearable device, or a home appliance. According to an embodiment of the disclosure, the electronic devices are not limited to those described above.

It should be appreciated that various embodiments of the present disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., wiredly), wirelessly, or via a third element.

As used in connection with various embodiments of the disclosure, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore^{TM}), or between two user devices (e.g., smart phones) directly. If distributed online, at least part of the computer program product may be temporarily generated or at least temporarily stored in the machine-readable storage medium, such as memory of the manufacturer’s server, a server of the application store, or a relay server.

According to various embodiments, each component (e.g., a module or a program) of the above-described components may include a single entity or multiple entities, and some of the multiple entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

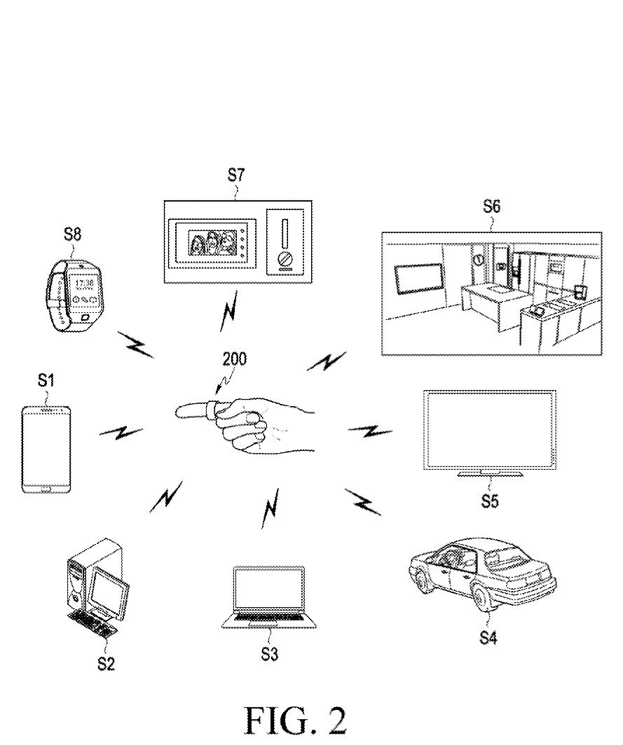

FIG. 2 is a view illustrating examples of use of a wearable electronic device according to an embodiment of the disclosure.

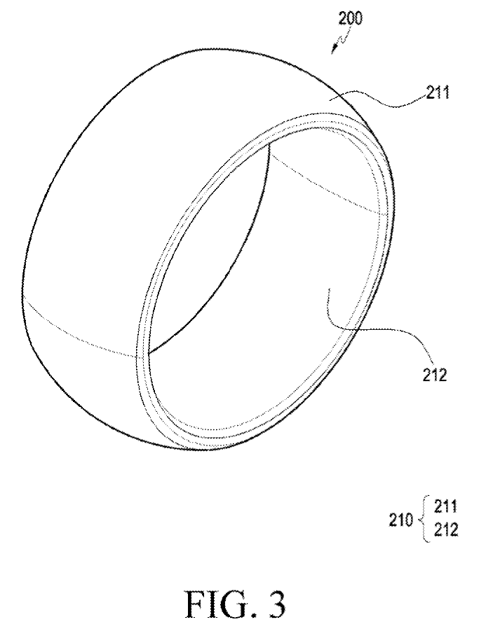

The embodiment of FIG. 2 may be combined with the embodiment of FIG. 1 or with the embodiments of FIGS. 3 to 13.

Referring to FIG. 2, a wearable electronic device 200 (e.g., the electronic device 101 of FIG. 1) may be configured to be wearable on a user’s body. For example, the wearable electronic device 200 may be implemented as a wearable electronic device that is wearable on a user’s finger. For example, the wearable electronic device 200 may be provided in the form of a ring that is wearable on a user’s finger or wrist. The wearable electronic device 200 may be defined and/or referred to as a smart ring.

According to an embodiment, the wearable electronic device 200 may perform wireless communication with another electronic device (e.g., the electronic device 102 or 104 of FIG. 1) through a wireless communication network (e.g., the first network 198 or the second network 199 of FIG. 1). For example, the wearable electronic device 200 may perform wireless communication with other electronic devices such as a smartphone S1, a desktop/laptop computer S2/S3, a vehicle S4, a smart TV S5, indoor smart home devices S6, a tablet PC S7, or a smartwatch S8. The wireless communication between the wearable electronic device 200 and another electronic device may be implemented through a short-range communication network (e.g., the first network 198 of FIG. 1) or a long-range communication network (e.g., the second network 199 of FIG. 1). For example, when a Bluetooth communication link is established between the wearable electronic device 200 and an electronic device to which a user desires to connect, message transmission between the two devices may be enabled. The wearable electronic device 200 worn by the user may generate commands corresponding to motions/gestures of the user’s finger and transmit the commands to another electronic device. To detect the user’s finger motions/gestures, the wearable electronic device 200 may include motion sensors such as an accelerometer, a gyroscope, or an electronic compass (e.g., the sensor module 176 of FIG. 1). When a message is received from another electronic device to the wearable electronic device 200, the electronic device 200 may notify the user of message reception using sound, vibration, a display screen, or lighting (e.g., a light-emitting diode or a xenon lamp). For this purpose, the wearable electronic device 200 may include an acoustic module (e.g., the sound output module 155 or the audio module 170 of FIG. 1), a haptic module (e.g., the haptic module 179 of FIG. 1), or a display module (e.g., the display module 160 of FIG. 1). According to an embodiment, in the electronic device, at least one of the acoustic module, the haptic module, or the display module may be omitted, or one or more other components may output information instead. In addition, the wearable electronic device 200 may acquire biometric information (e.g., oxygen saturation) of the user and provide the biometric information to another electronic device.

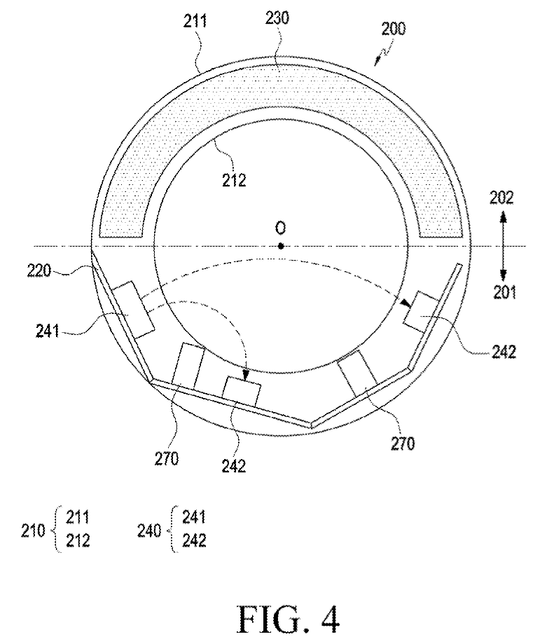

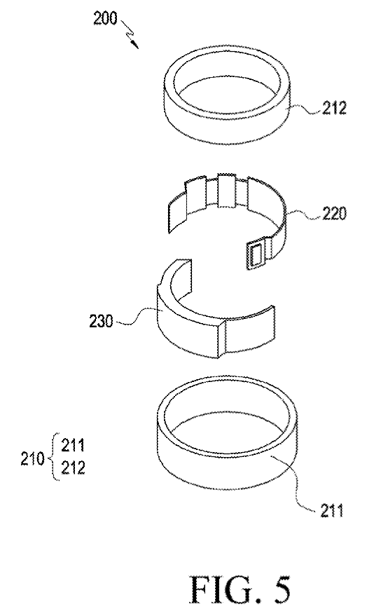

FIG. 3 is a perspective view illustrating a wearable electronic device according to an embodiment of the disclosure. FIG. 4 is a cross-sectional view of the wearable electronic device according to an embodiment of the disclosure. FIG. 5 is an exploded perspective view of the wearable electronic device according to an embodiment of the disclosure.

The embodiment of FIGS. 3 to 5 may be combined with the embodiments of FIGS. 1 and 2 or the embodiments of FIGS. 5 to 13. The configuration of the wearable electronic device 200 of FIGS. 3 to 5 may be partially or entirely the same as the configuration of the electronic device 101 of FIG. 1 or the configuration of the wearable electronic device 200 of FIG. 2.

Referring to FIGS. 3 to 5, the wearable electronic device 200 may include a housing 210. The housing 210 may form an overall external appearance of the wearable electronic device 200.

According to an embodiment, the housing 210 may be in a ring shape (e.g., a ring form). For example, the housing 210 may include an opening configured to accommodate a user’s finger. For example, the housing 210 may include an opening configured to accommodate a user’s wrist. For example, the opening may be defined as a hole formed at the housing 210. However, the wearable electronic device 200 is not limited to the ring-shaped embodiment described above and may be modified into various forms (e.g., a box shape) capable of being worn and/or fixed to a user’s body (e.g., finger, wrist).

According to an embodiment, the housing 210 may include an outer housing part 211 and an inner housing part 212. The inner housing part 212 may be coupled to the outer housing part 211. According to an embodiment, the outer housing part 211 and the inner housing part 212 may be separately manufactured and assembled or may be integrally formed.

According to an embodiment, the outer housing part 211 may include a material capable of withstanding external impact and/or scratches and implementing design characteristics. For example, the outer housing part 211 may include at least one of titanium, stainless steel, or ceramic. The outer housing part 211 may be color-treated or coating-treated for design implementation.

According to an embodiment, the inner housing part 212 may be a portion that comes into contact with a user’s finger when the user wears the wearable electronic device 200. The inner housing part 212 may be made of a material such as a molding material, transparent plastic, or glass for sensing. For example, the inner housing part 212 may be configured to be at least partially transparent. For example, the inner housing part 212 may include a material through which light for measuring biometric information is transmissible. At least a portion of the inner housing part 212 may be made of a material that is substantially the same as or similar to that of the outer housing part 211. In addition, at least a portion of the inner housing part 212 may include a metallic material for measuring biometric information.

According to an embodiment, the outer housing part 211 and the inner housing part 212 may be coupled to provide an internal space of the housing 210. Various electrical/electronic components of the wearable electronic device 200 may be disposed and/or mounted in the internal space of the housing 210. For example, the housing 210 may accommodate various electrical/electronic components.

According to an embodiment, the wearable electronic device 200 may include a circuit board 220, a battery 230, and at least one optical element 240, which are disposed within the housing 210.

According to an embodiment, the circuit board 220 may be disposed in the internal space of the housing 210. The circuit board 220 may include at least one of a printed circuit board (PCB), a flexible printed circuit board (FPCB), or a rigid-flexible PCB (RF-PCB).

According to an embodiment, various electrical/electronic components may be disposed and/or mounted on the circuit board 220. For example, the circuit board 220 may be provided with a processor (e.g., the processor 120 of FIG. 1), a memory (e.g., the memory 130 of FIG. 1), a communication module (e.g., the communication module 190 of FIG. 1), or a sensor module (e.g., the sensor module 176 of FIG. 1 or at least one optical element 240 of FIG. 4).

According to an embodiment, the circuit board 220 may include a plurality of printed circuit boards. For example, the plurality of printed circuit boards may be arranged along the shape of the internal space of the housing 210 and may be electrically connected to each other. The circuit board 220 may include a flexible printed circuit board (FPCB). For example, the flexible printed circuit board may be at least partially bent along the shape of the internal space of the housing 210. For example, the circuit board 220 may include rigid printed circuit boards and flexible printed circuit boards alternately arranged along a circumferential direction (or peripheral direction) of the housing 210.

According to an embodiment, the battery 230 may serve as a device configured to supply power to a component of the wearable electronic device 200, and may include a non-rechargeable primary battery, a rechargeable secondary battery, or a fuel cell. The battery 230 may be integrally disposed inside the wearable electronic device 200 or may be detachably disposed with respect to the wearable electronic device 200. According to an embodiment, the battery 230 may be formed as a single integrated battery or may include a plurality of separable batteries. The battery 230 may include a battery pack that is bendable along the shape of an internal space of the housing 210. The battery 230 may include a plurality of battery packs that are not bendable within the housing 210. The battery 230 may include a bendable battery pack or a plurality of non-bendable battery packs.

According to an embodiment, the circuit board 220 may be disposed in a first region 201 of the housing 210 (e.g., a region disposed at a lower side in FIG. 4). According to an embodiment, the battery 230 may be disposed in a second region 202 of the housing 210 (e.g., a region disposed at an upper side in FIG. 4) other than the first region 201 of the housing 210. For example, the housing 210 may be divided into the first region 201 and the second region 202 based on an imaginary reference line passing through a center O, and the battery 230 may be disposed in the second region 202 while the circuit board 220 may be disposed in the first region 201. The first region 201 and the second region 202 may be substantially the same in size and shape. The first region 201 and the second region 202 may be substantially symmetrical with respect to the center O of the housing 210.

According to an embodiment, the wearable electronic device 200 may include a power management module (e.g., the power management module 188 of FIG. 1) disposed on the circuit board 220.

According to an embodiment, the optical element 240 of the wearable electronic device 200 may be disposed within the housing 210 and may be electrically connected to the circuit board 220. According to an embodiment, the optical element 240 of the wearable electronic device 200 may include at least one light emitter 241 and at least one light receiver 242. According to an embodiment, the optical element 240 of the wearable electronic device 200 may include a sensor configured to acquire (or measure) at least one piece of biometric information. For example, the at least one piece of biometric information may include at least one of a user’s oxygen saturation information or a user’s heart rate information. For example, the sensor may include a photo-plethysmography (PPG) sensor configured to measure oxygen saturation or heart rate.

According to an embodiment, the PPG sensor may include a light source (e.g., at least one light emitter 241) configured to emit light in two wavelength bands (e.g., a red wavelength band or an infrared wavelength band). The PPG sensor may include a light-receiving unit (e.g., at least one light receiver 242) configured to detect at least a portion of light reflected from or transmitted through a user’s body part (e.g., a finger, or the skin or a blood vessel the finger).

According to an embodiment, the at least one light emitter 241 may radiate light to a user’s body part (e.g., a finger, or the skin and/or a blood vessel of the finger) by emitting light beams having substantially the same or different wavelengths, respectively, so as to measure a user’s oxygen saturation. For example, the at least one light emitter 241 may emit light beams in various wavelength bands and may include at least one of a light-emitting diode (LED), a laser diode, or a vertical cavity surface emitting laser (VCSEL). The at least one light emitter 241 may be disposed and/or mounted on the circuit board 220. The at least one light emitter 241 may be configured to sequentially (or repeatedly) emit light beams having different wavelength bands by dividing time.

According to an embodiment, the at least one light receiver 242 may accumulate photocharge corresponding to an amount of light incident to the at least one light receiver 242 after being reflected and/or transmitted on a user’s body part and may convert a biometric signal in an analog current form according to the accumulated photocharge into a digital signal. For example, light (or an optical signal) acquired (or detected) through the at least one light receiver 242 may be converted through an ADC (analog-to-digital converter) and stored in a memory or a sensor buffer. At least one light receiver 242 may include at least one of a photodiode (PD), a photo transistor, a charge-coupled device (CCD), or a complementary metal oxide semiconductor (CMOS). The at least one light receiver 242 may include, without being limited thereto, various elements capable of converting an incident optical signal into an electrical signal.

According to an embodiment, the at least one light receiver 242 may be configured to receive at least a portion of light transmitted through a user’s body part or at least a portion of light reflected by at least a portion of the user’s body part. The at least one light receiver 242 may be configured to receive light transmitted through a user’s body part, to convert the transmitted light into an electrical signal, and to transmit the electrical signal to a processor (e.g., the processor 120 of FIG. 1). According to an embodiment, the light receiver 242 may be configured to receive light reflected by at least a portion of a user’s body part. The light receiver 242 may be configured to receive light reflected by a user’s body part, to convert the reflected light into an electrical signal, and to transmit the electrical signal to a processor (e.g., the processor 120 of FIG. 1).

According to an embodiment, light emitted from the at least one light emitter 241 may reach the at least one light receiver 242 through a light path. For example, the light path may be a path through which light is transmitted through a user’s body part (e.g., a finger, or the skin or a blood vessel of the finger) or a path through which light is reflected by the user’s body part.

According to an embodiment, the wearable electronic device 200 may include at least one blocking member 270. The at least one blocking member 270 may include a material configured to absorb or block at least a portion of light emitted from the at least one light emitter 241. The at least one blocking member 270 may be configured to block propagation of light emitted from the at least one light emitter 241 within an internal space of the housing 210. For example, the at least one blocking member 270 may be disposed adjacent to the light receiver 242 within the internal space of the housing 210. For example, the at least one blocking member 270 may be positioned between the light receiver 242 and the light emitter 241 within the internal space of the housing 210.

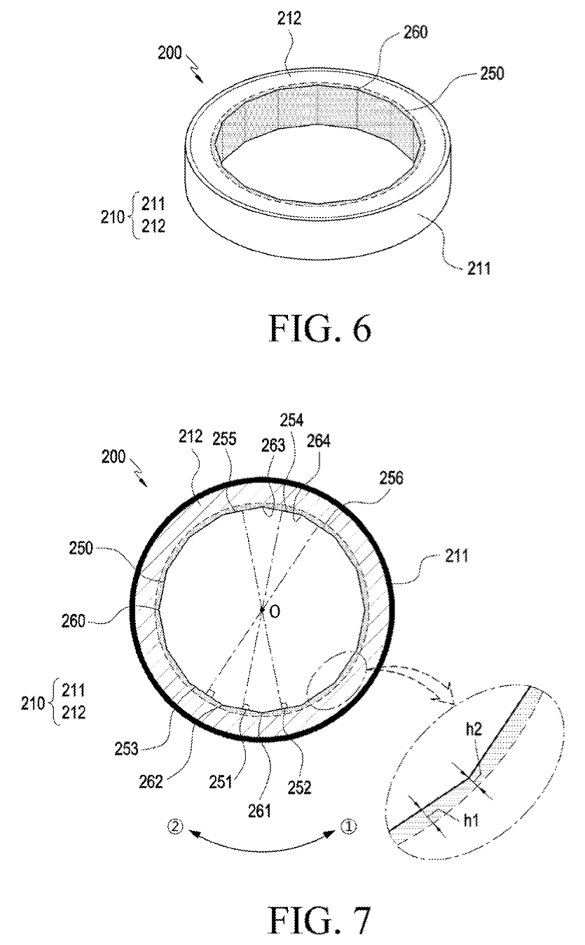

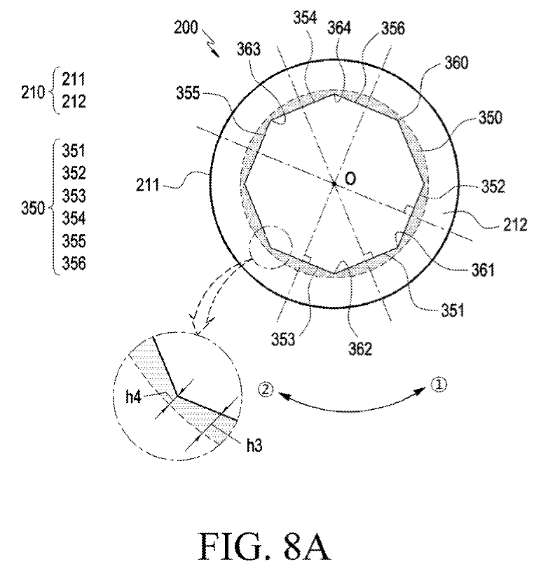

FIG. 6 is a perspective view of a wearable electronic device 200 according to an embodiment of the disclosure. FIG. 7 is a side view of the wearable electronic device 200 according to an embodiment of the disclosure.

Referring to FIGS. 6 and 7, the wearable electronic device 200 may include a housing 210 including an outer housing part 211 and an inner housing part 212, and a plurality of protrusions 250 disposed on the inner housing part 212. The configuration of the wearable electronic device 200 illustrated in FIGS. 6 and 7 may be entirely or partially the same as the configuration of the wearable electronic device 200 illustrated in FIGS. 3 to 5. The embodiment of FIGS. 6 and 7 may be partially combined with the embodiment of FIGS. 3 to 5. According to FIGS. 6 and 7, the plurality of protrusions 250 may be defined as shaded gray portions.

According to an embodiment, the plurality of protrusions 250 may press a vascular region of a finger to surely secure a blood vessel when emitting or receiving light toward the vascular region of the finger.

According to an embodiment, the plurality of protrusions 250 may be disposed on the inner housing part 212. For example, the plurality of protrusions 250 may be arranged to protrude toward the center O from the inner housing part 212. According to an embodiment, the plurality of protrusions 250 may be formed to protrude inwardly relative to the inner housing part 212.

According to an embodiment, the plurality of protrusions 250 may be integrally formed with the inner housing part 212. The plurality of protrusions 250 may be made of a material such as a molding material, transparent plastic, or glass for sensing. For example, the plurality of protrusions 250 may be at least partially transparent. For example, the plurality of protrusions 250 may include a material through which light for measuring biometric information is transmissible. For example, the transmissive material may be made of a material that allows at least a portion of light emitted from at least one light emitter 241 for measuring biometric information to transmit therethrough, or allows at least a portion of light reflected from a portion of the user’s body for acquiring the biometric information to be transmitted therethrough. At least a portion of the plurality of protrusions 250 may be made of a material that is substantially the same as or similar to that of the inner housing part 212 and/or the outer housing part 211. In addition, at least a portion of the plurality of protrusions 250 may include a metallic material for measuring biometric information.

According to an embodiment, the plurality of protrusions 250 may be continuously arranged along one surface (e.g., the inner circumferential surface) of the inner housing part 212. For example, the plurality of protrusions 250 may be arranged in a ring shape along the circumferential direction (e.g., the peripheral direction) of the inner housing part 212.

According to an embodiment, the plurality of protrusions 250 may be continuously arranged in a first direction (e.g., a counterclockwise direction) and/or in a second direction (e.g., a clockwise direction) opposite to the first direction. For example, the plurality of protrusions 250 may include a first protrusion 251, a second protrusion 252 positioned in the first direction of the first protrusion 251 and extending from the first protrusion 251, and a third protrusion 253 positioned in the second direction opposite to the first direction of the first protrusion 251 and extending from the first protrusion 251. The first direction may be a first circumferential direction. The second direction may be a second circumferential direction opposite to the first circumferential direction.

According to an embodiment, the plurality of protrusions 250 may be symmetrically arranged with respect to the center O of the housing. According to an embodiment, by symmetrically arranging the plurality of protrusions 250, the vascular region of a finger may be easily secured by pressing the finger from all directions relative to the finger, while also providing an aesthetic function. For example, the plurality of protrusions 250 may include a fourth protrusion 254 symmetrically arranged with the first protrusion 251 with respect to the center, a fifth protrusion 255 symmetrically arranged with the second protrusion 252 with respect to the center, and a sixth protrusion 256 symmetrically arranged with the third protrusion 253 with respect to the center. The fifth protrusion 255 may be positioned in the first direction of the fourth protrusion 254 and may extend from the fourth protrusion 254. The sixth protrusion 256 may be positioned in the second direction of the fourth protrusion 254 and may extend from the fourth protrusion 254.

According to an embodiment, the plurality of protrusions 250 may be formed such that each of the lengths thereof from inner surface of the inner housing part 212 increases toward a central portion rather than an edge portion. According to an embodiment, the central portion of at least one protrusion 250 may be defined as a portion having the greatest distance from inner surface of the inner housing part 212. According to an embodiment, at least one protrusion 250 may be formed such that its length from inner surface of the inner housing part 212 increases toward a central portion rather than an edge portion, and may protrude toward the center O of the wearable electronic device 200. According to an embodiment, a vertical distance from inner surface of the inner housing part 212 to a central portion of the at least one protrusion 250 may be defined as a first length h1. According to an embodiment, a vertical distance from inner surface of the inner housing part 212 to an edge portion of the at least one protrusion may be defined as a second length h2. For example, the second length h2 may be zero or greater than zero. For example, the second length h2 may be smaller than the first length h1. According to an embodiment, central portions of the plurality of protrusions 250 may have a larger contact area with a user’s finger. According to an embodiment, edge portions of the plurality of protrusions 250 may have a relatively smaller contact area with a user’s finger or may not come into contact with the user’s finger. According to an embodiment, a plurality of recess regions 260 may be formed between respective protrusions constituting the plurality of protrusions 250. Recess regions 260 may be formed at edge portions according to a height difference between a central portion and an edge portion of each of the plurality of protrusions 250. For example, a first recess region 261 may be formed between the first protrusion 251 and the second protrusion 252. For example, a second recess region 262 may be formed between the first protrusion 251 and the third protrusion 253. For example, a third recess region 263 may be formed between the fourth protrusion 254 and the fifth protrusion 255. For example, a fourth recess region 264 may be formed between the fourth protrusion 254 and the sixth protrusion 256.

According to an embodiment, the plurality of protrusions 250 may have various shapes. For example, the plurality of protrusions 250 may be disposed on the inner housing part 212, and one surface of each protrusion oriented toward the center may be planar. For example, the surface oriented toward the center may be arranged perpendicularly to a reference line passing through the center O.

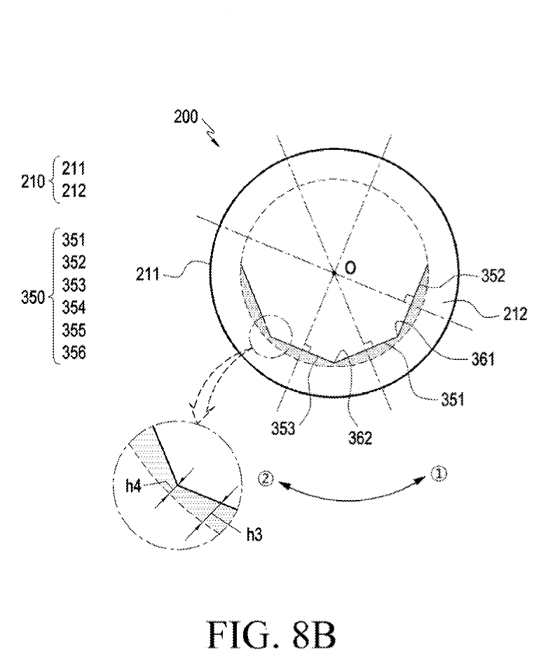

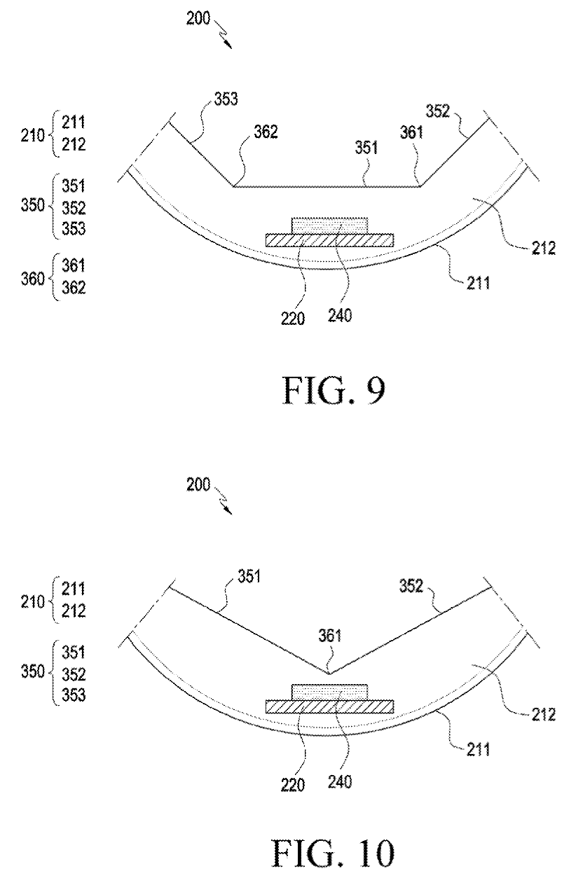

FIGS. 8A and 8B are side views of a wearable electronic device 200 according to an embodiment of the disclosure. FIGS. 9 and 10 are views illustrating a portion of an internal space of the wearable electronic device 200 according to an embodiment of the disclosure.

Referring to FIGS. 8A to 10, the wearable electronic device 200 may include a housing 210 including an outer housing part 211 and an inner housing part 212, and a plurality of protrusions 350 disposed on the inner housing part 212. The configuration of the wearable electronic device 200 illustrated in FIGS. 8A to 10 may be entirely or partially the same as that of the wearable electronic device 200 illustrated in FIGS. 3 to 7. The embodiment illustrated in FIGS. 8A to 10 may be partially combined with the embodiments illustrated in FIGS. 3 to 7. According to FIGS. 8A and 8B, the plurality of protrusions 350 may be defined as gray-shaded portions.

According to an embodiment, the plurality of protrusions 350 may be continuously arranged along one surface (e.g., the inner circumferential surface) of the inner housing part 212. For example, the plurality of protrusions 350 may be arranged in a ring shape along the circumferential direction (e.g., the peripheral direction).

According to an embodiment, the plurality of protrusions 350 may be continuously arranged in a first direction (e.g., a counterclockwise direction) and/or in a second direction (e.g., a clockwise direction) opposite to the first direction. For example, the plurality of protrusions 350 may include a first protrusion 351, a second protrusion 352 positioned in the first direction of the first protrusion 351 and extending from the first protrusion 351, and a third protrusion 353 positioned in the second direction opposite to the first direction of the first protrusion 351 and extending from the first protrusion 351.

According to an embodiment, the plurality of protrusions 350 may be symmetrically arranged with respect to the center O of the housing. According to an embodiment, by symmetrically arranging the plurality of protrusions 350, the vascular region of a finger may be easily secured by pressing the finger from all directions relative to the finger, while also providing an aesthetic function. For example, the plurality of protrusions 350 may include a fourth protrusion 354 symmetrically arranged with the first protrusion 351 with respect to the center, a fifth protrusion 355 symmetrically arranged with the second protrusion 352 with respect to the center, and a sixth protrusion 356 symmetrically arranged with the third protrusion 353 with respect to the center. The fifth protrusion 355 may be positioned in the first direction of the fourth protrusion 354 and may extend from the fourth protrusion 354. The sixth protrusion 356 may be positioned in the second direction of the fourth protrusion 354 and may extend from the fourth protrusion 354. However, the arrangement of the plurality of protrusions 350 is not limited to the above embodiments and may be variously modified in design. According to an embodiment, referring to FIG. 8B, the plurality of protrusions 350 may be asymmetrically arranged with respect to the center O of the housing. According to an embodiment, referring to FIG. 8B, the plurality of protrusions 350 may be asymmetrically arranged with respect to the center O of the housing. For example, the plurality of protrusions 350 may include a first protrusion 351, a second protrusion 352 positioned in the first direction of the first protrusion 351 and extending from the first protrusion 351, and a third protrusion 353 positioned in the second direction opposite to the first direction of the first protrusion 351 and extending from the first protrusion 351. For example, the plurality of protrusions 350 may not include protrusions that are symmetrically arranged with respect to the center with the first protrusion 351, the second protrusion 352, or the third protrusion 353. For example, the plurality of protrusions 350 may not include protrusions continuously arranged in the first direction from the second protrusion 352. For example, the plurality of protrusions 350 may not include protrusions continuously arranged in the second direction from the third protrusion 353.

According to an embodiment, at least one protrusion 350 may be formed such that its length from inner surface of the inner housing part 212 increases toward a central portion rather than an edge portion. According to an embodiment, the central portion of at least one protrusion 350 may be defined as a portion having the greatest distance from inner surface of the inner housing part 212. According to an embodiment, at least one protrusion 350 may be formed such that its length from inner surface of the inner housing part 212 increases toward a central portion rather than an edge portion, and may protrude toward the center O of the wearable electronic device 200. According to an embodiment, a vertical distance from inner surface of the inner housing part 212 to a central portion of the at least one protrusion 350 may be defined as a third length h3. According to an embodiment, a vertical distance from inner surface of the inner housing part 212 to an edge portion of the at least one protrusion may be defined as a fourth length h4. For example, the fourth length h4 may be zero or greater than zero. For example, the fourth length h4 may be smaller than the third length h3. According to an embodiment, central portions of the plurality of protrusions 350 may have a larger contact area with a user’s finger. According to an embodiment, edge portions of the plurality of protrusions 350 may have a relatively smaller contact area with a user’s finger or may not come into contact with the user’s finger.

According to an embodiment, a plurality of recess regions 360 may be formed between respective protrusions constituting the plurality of protrusions 350. Recess regions 360 may be formed at edge portions according to a height difference between a central portion and an edge portion of each of the plurality of protrusions 350. For example, a first recess region 361 may be formed between the first protrusion 351 and the second protrusion 352. For example, a second recess region 362 may be formed between the first protrusion 351 and the third protrusion 353. For example, a third recess region 363 may be formed between the fourth protrusion 354 and the fifth protrusion 355. For example, a fourth recess region 364 may be formed between the fourth protrusion 354 and the sixth protrusion 356.

According to an embodiment, the plurality of protrusions 350 may have various shapes. For example, the plurality of protrusions 350 may be disposed on the inner housing part 212, and at least a portion of a surface of each protrusion oriented toward the center may be planar. For example, the surface oriented toward the center may be arranged substantially perpendicularly to a reference line passing through the center O.

According to an embodiment, the number of the plurality of protrusions 350 may vary. For example, referring to FIG. 7, there may be sixteen protrusions 250. For example, referring to FIG. 8A, there may be eight protrusions 350. However, the number of protrusions 350 is not limited to the above embodiments and may be variously modified in design.

According to an embodiment, the sizes and shapes of the plurality of protrusions 350 may be substantially the same. According to an embodiment, the sizes and shapes of the plurality of protrusions 350 may be different from each other. For example, protrusions having a first size and protrusions having a second size may be alternately arranged. However, the sizes and shapes of the plurality of protrusions 350 are not limited to the above embodiments and may be variously modified in design.

According to an embodiment, the at least one optical element 240 may be arranged to face at least one of the plurality of protrusions 350. For example, referring to FIG. 9, the at least one optical element 240 may be arranged to face the first protrusion 351. The at least one optical element 240 may be disposed parallel to a planar portion of the first protrusion 351. When the optical element 240 faces the first protrusion 351, light emitted from the optical element 240 may vertically pass through the first protrusion 351, which may result in a higher amount of incident light at the first protrusion 351. When the optical element 240 faces the first protrusion 351, since the incident light amount on the optical element 240 from the first protrusion 351 is maximized, the amount of light reflected from or transmitted through a user’s body part (e.g., a finger, or the skin or a blood vessel of the finger) may be relatively large.

According to an embodiment, the at least one optical element 240 may be arranged to face at least one of the plurality of recess regions 360. For example, referring to FIG. 10, the at least one optical element 240 may be arranged to face a first recess region 361. The first recess region 361 may be positioned closer to the optical element 240 than the first protrusion 351. When the optical element 240 faces the first recess region 361, light emitted from the optical element 240 may be concentrated on a localized area of the first recess region 361, which may result in a higher amount of incident light at the first recess region 361. Since a finger protrudes into the first recess region 361, the amount of light reflected from or transmitted through a user’s body part (e.g., a finger, or the skin or a blood vessel of the finger) may be relatively large.

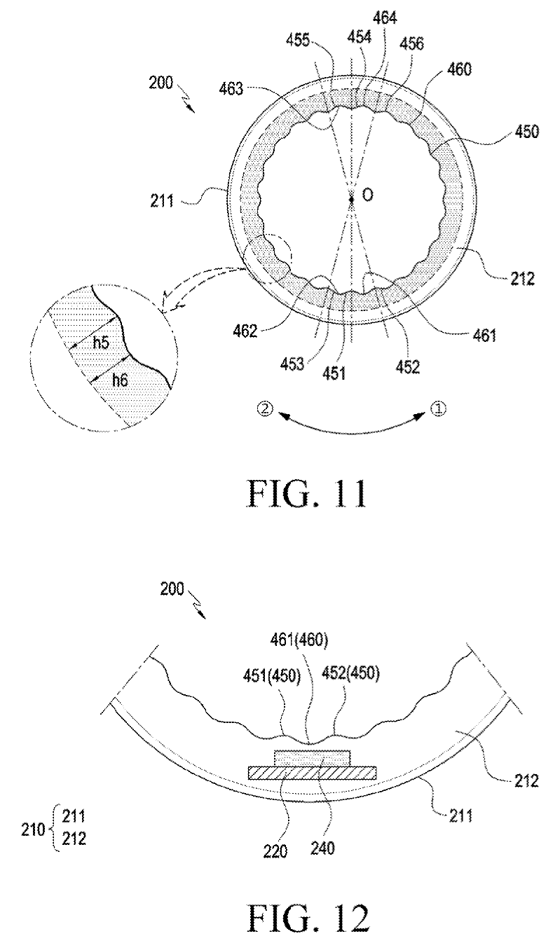

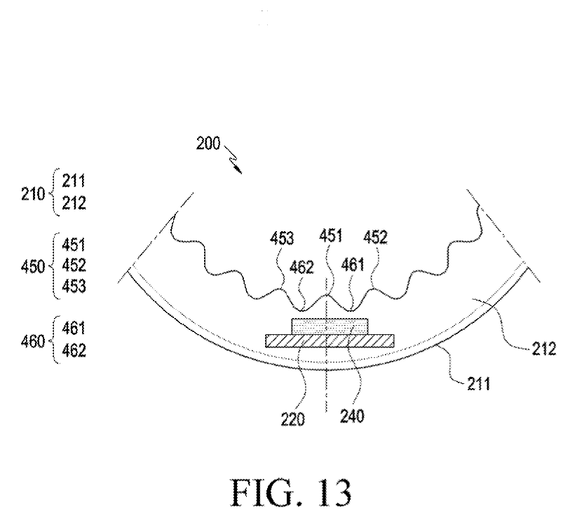

FIG. 11 is a side view of a wearable electronic device 200 according to an embodiment of the disclosure. FIGS. 12 and 13 are views illustrating a portion of an internal space of the wearable electronic device 200 according to an embodiment of the disclosure.

Referring to FIGS. 11 to 13, the wearable electronic device 200 may include a housing 210 including an outer housing part 211 and an inner housing part 212, and a plurality of protrusions 450 disposed on the inner housing part 212. The configuration of the wearable electronic device 200 illustrated in FIGS. 11 to 13 may be entirely or partially the same as that of the wearable electronic device 200 illustrated in FIGS. 3 to 10. The embodiment illustrated in FIGS. 11 to 13 may be partially combined with the embodiments illustrated in FIGS. 3 to 10.

According to an embodiment, the plurality of protrusions 450 may be continuously arranged along one surface (e.g., the inner circumferential surface) of the inner housing part 212. For example, the plurality of protrusions 450 may be arranged in a ring shape along the circumferential direction (e.g., the peripheral direction).

According to an embodiment, the plurality of protrusions 450 may be continuously arranged in a first direction (e.g., a counterclockwise direction) and/or in a second direction (e.g., a clockwise direction) opposite to the first direction. For example, the plurality of protrusions 450 may include a first protrusion 451, a second protrusion 452 positioned in the first direction of the first protrusion 451 and extending from the first protrusion 451, and a third protrusion 453 positioned in the second direction opposite to the first direction of the first protrusion 451 and extending from the first protrusion 451.

According to an embodiment, the plurality of protrusions 450 may be symmetrically arranged with respect to the center O of the housing. According to an embodiment, by symmetrically arranging the plurality of protrusions 450, the vascular region of a finger may be easily secured by pressing the finger from all directions relative to the finger, while also providing an aesthetic function. For example, the plurality of protrusions 450 may include a fourth protrusion 454 symmetrically arranged with the first protrusion 451 with respect to the center, a fifth protrusion 455 symmetrically arranged with the second protrusion 452 with respect to the center, and a sixth protrusion 456 symmetrically arranged with the third protrusion 453 with respect to the center. The fifth protrusion 455 may be positioned in the first direction of the fourth protrusion 454 and may extend from the fourth protrusion 454. The sixth protrusion 456 may be positioned in the second direction of the fourth protrusion 454 and may extend from the fourth protrusion 454.

According to an embodiment, the plurality of protrusions 450 may be formed such that each of the lengths thereof from inner surface of the inner housing part 212 increases toward a central portion rather than an edge portion. According to an embodiment, the central portions of the plurality of protrusions 450 may be defined as portions having the greatest distance from inner surface of the inner housing part 212. According to an embodiment, the plurality of protrusions 450 may be formed such that the lengths from inner surface of the inner housing part 212 increase toward central portions rather than edge portions, and may protrude toward the center O of the wearable electronic device 200. According to an embodiment, a vertical distance from inner surface of the inner housing part 212 to a central portion of each of the plurality of protrusions 450 may be a fifth length h5. According to an embodiment, a vertical distance from inner surface of the inner housing part 212 to an edge portion of the at least one protrusion may be defined as a sixth length h6. For example, the sixth length h6 may be zero or may be greater than zero. For example, the sixth length h6 may be smaller than the fifth length h5. According to an embodiment, central portions of the plurality of protrusions 450 may have a larger contact area with a user’s finger. According to an embodiment, edge portions of the plurality of protrusions 450 may have a relatively smaller contact area with a user’s finger or may not come into contact with the user’s finger.

According to an embodiment, a plurality of recess regions 460 may be formed between respective protrusions constituting the plurality of protrusions 450. Recess regions 460 may be formed at edge portions according to a height difference between a central portion and an edge portion of each of the plurality of protrusions 450. For example, a first recess region 461 may be formed between the first protrusion 451 and the second protrusion 452. For example, a second recess region 462 may be formed between the first protrusion 451 and the third protrusion 453. For example, a third recess region 463 may be formed between the fourth protrusion 454 and the fifth protrusion 455. For example, a fourth recess region 464 may be formed between the fourth protrusion 454 and the sixth protrusion 456.

According to an embodiment, the plurality of protrusions 450 may have various shapes. For example, the plurality of protrusions 450 may be disposed on the inner housing part 212, and one surface of each of the protrusions oriented toward the center O may be a curved surface protruding inward. For example, the one surface oriented toward the center O may be arranged perpendicularly to a reference line passing through the center O.

According to an embodiment, the sizes and shapes of the plurality of protrusions 450 may be substantially the same. According to an embodiment, the sizes and shapes of the plurality of protrusions 450 may be different from each other. For example, protrusions having a first size and protrusions having a second size may be alternately arranged. However, the sizes and shapes of the plurality of protrusions 450 are not limited to the above embodiments and may be variously modified in design.

According to an embodiment, the at least one optical element 240 may be arranged to face at least one of the plurality of protrusions 450. For example, referring to FIG. 13, the at least one optical element 240 may be arranged to face a first protrusion 451 at the center. When the optical element 240 faces the first protrusion 451, since the incident light amount on the optical element 240 from the first protrusion 451 is maximized, the amount of light reflected from or transmitted through a user’s body part (e.g., a finger, or the skin or a blood vessel of the finger) may be relatively large.

According to an embodiment, the at least one optical element 240 may be arranged to face at least one of the plurality of recess regions 460. For example, referring to FIG. 12, the at least one optical element 240 may be arranged to face a first recess region 461 at the center. When the optical element 240 faces the first recess region 461, the finger may protrude into the first recess region 461, so that the amount of light reflected from or transmitted through a user’s body part (e.g., a finger, or a blood vessel or the skin of the finger) may be relatively large.

An electronic device according to an embodiment of the disclosure may include a housing including an outer housing part and an inner housing part coupled to the outer housing part, a circuit board disposed within the housing, at least one optical element disposed within the housing and electrically connected to the circuit board, and a plurality of protrusions arranged along inner surface of the inner housing part. The at least one optical element may be arranged to face at least one of the plurality of protrusions.

According to an embodiment, a plurality of recess regions 260, 360, and 460 may be formed between respective protrusions constituting the plurality of protrusions.

According to an embodiment, a vertical distance from a central portion of each of the plurality of protrusions to the inner surface of the inner housing part may be defined as a first length h1, and a vertical distance from an edge portion of each of the plurality of protrusions to the inner surface of the inner housing part may be defined as a second length h2 shorter than the first length h1.

According to an embodiment, the plurality of protrusions and the inner housing part may be integrally formed.

According to an embodiment, the plurality of protrusions may be configured to be at least partially transparent.

According to an embodiment, the plurality of protrusions may include a first protrusion 251, 351, or 451, a second protrusion 252, 352, 452 positioned in a first direction from the first protrusion and extending from the first protrusion, and a third protrusion 253, 353, or 453 positioned in a second direction opposite to the first direction from the first protrusion and extending from the first protrusion.

According to an embodiment, the plurality of protrusions may further include a fourth protrusion 254, 354, or 454 arranged symmetrically with the first protrusion about a center, a fifth protrusion 255, 355, or 455 arranged symmetrically with the second protrusion with respect to the center and positioned in the first direction of the fourth protrusion, and a sixth protrusion 256, 356, or 456 arranged symmetrically with the third protrusion with respect to the center and positioned in the second direction of the fourth protrusion.

According to an embodiment, one surface of each of the plurality of protrusions 250 and 350 oriented toward a center may be a flat surface.

According to an embodiment, one surface of each of the plurality of protrusions 450 oriented toward the center may be a curved surface.

According to an embodiment, the circuit board may include rigid printed circuit boards and flexible printed circuit boards alternately arranged.

According to an embodiment, the electronic device may further include a battery 230 disposed within the housing, and the housing may be divided into a first region and a second region, in which the battery may be disposed in the second region and the circuit board may be disposed in the first region.

According to an embodiment, the electronic device may further include a processor disposed within the housing, and the at least one optical element may include at least one light emitter 241 configured to emit light through the inner housing part and at least one light receiver 242 configured to receive light transmitted through or reflected from a user’s finger, and the processor may be configured to acquire at least one piece of biometric information by using a signal detected through the at least one light receiver.

According to an embodiment, the at least one piece of biometric information may include at least one of oxygen saturation information and heart rate information.

According to an embodiment, the electronic device may further include at least one blocking member 270 disposed within the housing and configured to absorb or block light.

According to an embodiment, the at least one light emitter may be configured to emit light in a plurality of wavelength bands, and the plurality of wavelength bands may include a red wavelength band and an infrared wavelength band.

An electronic device according to an embodiment of the disclosure may include a housing including an outer housing part and an inner housing part coupled to the outer housing part, a circuit board disposed within the housing, at least one optical element disposed within the housing and electrically connected to the circuit board, and a plurality of protrusions continuously arranged along inner surface of the inner housing part and symmetrically disposed with respect to the center of the housing. A plurality of recess regions may be formed between respective protrusions constituting the plurality of protrusions. The at least one optical element may be arranged to face at least one of the plurality of protrusions or the plurality of recess regions.

According to an embodiment, a vertical distance from a central portion of each of the plurality of protrusions to the inner surface of the inner housing part may be defined as a first length h1, and a vertical distance from an edge portion of each of the plurality of protrusions to the inner surface of the inner housing part may be defined as a second length h2 shorter than the first length h1.

According to an embodiment, the plurality of protrusions and the inner housing part may be integrally formed.

According to an embodiment, the plurality of protrusions may be configured to be at least partially transparent.

According to an embodiment, the plurality of protrusions may include a first protrusion, a second protrusion positioned in the first direction from the first protrusion and extending from the first protrusion, and a third protrusion positioned in a second direction opposite to the first direction from the first protrusion and extending from the first protrusion.

A ring-shaped wearable electronic device may emit or receive light to or from a blood vessel region of a user’s finger in order to obtain health data, and a protrusion having a convex shape may be disposed on the blood vessel region of the finger to secure the blood vessel. However, when the protrusion is arranged to correspond to the optical sensor, pressure may be applied to only one side, resulting in discomfort in wearing.

The electronic device according to an embodiment of the disclosure may include a plurality of protrusions symmetrically arranged with respect to the center of the housing, thereby allowing the finger to be evenly pressed in all directions and improving wearing comfort and aesthetics.

An electronic device according to an embodiment of the disclosure may include a housing having a ring shaped and comprising an outer housing part and an inner housing part, wherein the inner housing part coupled to the outer housing part, a circuit board disposed within the housing, at least one optical element disposed within the housing, the at least one optical element being electrically connected to the circuit board and a plurality of protrusions arranged along an inner circumferential surface of the inner housing part. The at least one optical element may be arranged to face at least one of the plurality of protrusions.

According to an embodiment, each protrusion of the plurality of protrusions may comprise a central portion and an edge portion,

According to an embodiment, a vertical distance from the central portion to the inner circumferential surface of the inner housing part may be greater than a vertical distance from the edge portion to the inner circumferential surface of the inner housing part.

According to an embodiment, each protrusion among the plurality of protrusions may comprise a flat surface oriented toward the center of the housing.

According to an embodiment, each protrusion among the plurality of protrusions may comprise a curved surface oriented toward the center of the housing.

An electronic device according to an embodiment of the disclosure may comprise a housing comprising an outer housing part and an inner housing part, the inner housing part being coupled to the outer housing part; a circuit board disposed within the housing; at least one optical element disposed within the housing and electrically connected to the circuit board; and a plurality of protrusions arranged along an inner circumferential surface of the inner housing part and arranged symmetrically with respect to a center of the housing. The plurality of recess regions may be provided between adjacent protrusions of the plurality of protrusions. And the at least one optical element may face at least one protrusion of the plurality of protrusions or the plurality of recess regions.

However, the problems to be solved by the disclosure are not limited to those mentioned above, and may be variously extended without departing from the spirit and scope of the disclosure.

What is claimed is:

1. An electronic device comprising:
a housing having a ring shape and comprising an outer housing part and an inner housing part, the inner housing part being coupled to the outer housing part;
a circuit board disposed within the housing;
at least one optical element disposed within the housing, the at least one optical element being electrically connected to the circuit board; and
a plurality of protrusions arranged along an inner circumferential surface of the inner housing part,
wherein the at least one optical element faces at least one protrusion of the plurality of protrusions.

2. The electronic device of claim 1, wherein a plurality of recess regions are provided between adjacent protrusions of the plurality of protrusions.

3. The electronic device of , wherein each protrusion of the plurality of protrusions comprises a central portion and an edge portion, and a vertical distance from the central portion to the inner circumferential surface of the inner housing part is greater than a vertical distance from the edge portion to the inner circumferential surface of the inner housing part.

4. The electronic device of claim 1, wherein the plurality of protrusions are integrally formed with the inner housing part .

5. The electronic device of claim 1, wherein the plurality of protrusions are at least partially transparent.

6. The electronic device of claim 1, wherein the plurality of protrusions comprise:
a first protrusion;
a second protrusion positioned in a first circumferential direction from the first protrusion and extending from the first protrusion; and
a third protrusion positioned in a second circumferential direction and extending from the first protrusion, the second circumferential direction being opposite to the first circumferential direction.

7. The electronic device of claim 6, wherein the plurality of protrusions further comprise:
a fourth protrusion arranged symmetrically with the first protrusion with respect to a center of the housing;
a fifth protrusion arranged symmetrically with the second protrusion with respect to the center of the housing and positioned in the first circumferential direction from the fourth protrusion; and
a sixth protrusion arranged symmetrically with the third protrusion with respect to the center of the housing and positioned in the second circumferential direction from the fourth protrusion.

8. The electronic device of claim 7, wherein each protrusion among the plurality of protrusions comprises a flat surface oriented toward the center of the housing.

9. The electronic device of claim 7, wherein each protrusion among the plurality of protrusions comprises a curved surface oriented toward the center of the housing.

10. The electronic device of claim 1, wherein the circuit board comprises rigid printed circuit boards and flexible printed circuit boards that are alternately arranged.

11. The electronic device of claim 1, further comprising:
a battery disposed within the housing,
wherein the housing comprises a first region and a second region, the battery is disposed in the first region, and the circuit board is disposed in the second region.

12. The electronic device of claim 1, further comprising:
a processor disposed within the housing,
wherein the at least one optical element comprises:
at least one light emitter configured to emit light through the inner housing part, and
at least one light receiver configured to receive light transmitted through or reflected from a user’s finger, and
wherein the processor is configured to obtain at least one biometric data based on a signal detected by the at least one light receiver.

13. The electronic device of claim 12, wherein the at least one biometric data comprises at least one of oxygen saturation data or heart rate data.

14. The electronic device of claim 1, further comprising:
at least one blocking member disposed within the housing and configured to absorb or block light.

15. The electronic device of claim 12, wherein the at least one light emitter is configured to emit light in a plurality of wavelength bands, and
wherein the plurality of wavelength bands comprise a red wavelength band and an infrared wavelength band.

16. An electronic device comprising:
a housing comprising an outer housing part and an inner housing part, the inner housing part being coupled to the outer housing part;
a circuit board disposed within the housing;
at least one optical element disposed within the housing and electrically connected to the circuit board; and
a plurality of protrusions arranged along an inner circumferential surface of the inner housing part and arranged symmetrically with respect to a center of the housing;
wherein a plurality of recess regions are provided between adjacent protrusions of the plurality of protrusions, and
wherein the at least one optical element faces at least one protrusion of the plurality of protrusions or the plurality of recess regions.

17. The electronic device of claim 16,
wherein each protrusion of the plurality of protrusions comprises a central portion and an edge portion, and a vertical distance from the central portion to the inner circumferential surface of the inner housing part is greater than a vertical distance from the edge portion to the inner circumferential surface of the inner housing part.

18. The electronic device of claim 16, wherein the plurality of protrusions are integrally formed with the inner housing part .

19. The electronic device of claim 16, wherein the plurality of protrusions are at least partially transparent.

20. The electronic device of claim 16,
wherein the plurality of protrusions comprise:
a first protrusion;
a second protrusion positioned in a first circumferential direction from the first protrusion and extending from the first protrusion; and
a third protrusion positioned in a second circumferential direction opposite to the first circumferential direction and extending from the first protrusion.
